(19) United States
(12) Patent Application Publication
(10) Pub. No.: US 2024/0065583 A1
(43) Pub. Date: Feb. 29, 2024

(54) SMART AUDIOMETER FOR AUDIOMETRIC TESTING

(71) Applicant: SOUNDTRACE LLC, Liberty Township, OH (US)

(72) Inventors: Jeffrey Wilson, Liberty Township, OH (US); Kevin Kast, Cincinnati, OH (US); Ryan Kast, Cincinnati, OH (US); Matt Reinhold, Cincinnati, OH (US)

(21) Appl. No.: 18/240,833

(22) Filed: Aug. 31, 2023

Related U.S. Application Data
(60) Provisional application No. 63/402,590, filed on Aug. 31, 2022.

(51) Int. Cl. A61B 5/12; A61B 5/00; G16H 15/00; G16H 40/67

(52) U.S. Cl. CPC A61B 5/123; A61B 5/0022; A61B 5/74; G16H 15/00; G16H 40/67; A61B 2560/0242

(57) ABSTRACT

A hearing health monitoring system, noise mitigating system, and method for monitoring hearing health are provided.

### PRIORITY

This application claims the benefit of U.S. Pat. App. No. 63/402,590, entitled “Smart Audiometer for Audiometric Testing,” filed Aug. 31, 2022, the disclosure of which is incorporated by reference herein.

### FIELD OF THE INVENTION

The invention relates generally to an instrument and software system that performs, processes, evaluates, retains, diagnoses, and predicts hearing acuity test results. Unlike traditional audiometric testing, this invention may provide a proactive and personalized method that integrates actual noise exposure and other contributing elements to calculate an accurate hearing level and predict a timeline for hearing loss decline. Additional applications through the use of an Audio Digital Signal Processor and software infrastructure also apply.

### BACKGROUND

The Centers for Disease Control and Prevention (CDC) has estimated that twenty-two million United States workers are exposed to hazardous noise levels annually, causing hearing loss to be one of the most common work-related illnesses. Furthermore, it is estimated that there are over 40 million Americans between the ages of 20-69 who suffer from Noise Induced Hearing Loss (NIHL). In this regard, the average person is born with about 16,000 hair cells within the inner ear, which allow the person's brain to detect sounds. By the time a person experiencing hearing loss notices a loss of hearing, many hair cells have already been damaged or destroyed. In some instances, a person experiencing hearing loss may lose 30% to 50% of hair cells within the inner ear before loss of hearing can be measured by a hearing test. Damaged inner ear hair cells typically do not grow back, thereby making noise induced hearing loss a permanent injury as there is no present cure.

Damage to inner ear hair cells can also cause damage to the auditory nerve that carries information about sounds to the brain. Hearing loss can also lead to other health effects such as tinnitus, depression, anxiety, high blood pressure, dementia and other health, social and physiological impacts. Noise induced hearing loss for workers can result in lost wages, lost ability to work and other lifetime challenges, causing an estimate of over $242 million in annual workers' compensation settlements and expensive fines by the Occupational Safety & Health Administration (OSHA). In the United States alone, hearing loss has an annual economic impact of $133 billion. This is due to loss of productivity, underemployment, unemployment, early retirement, healthcare and other related costs.

NIHL is the only type of hearing loss that is completely preventable. By understanding the hazards of noise and implementing early identification and intervention with corrective actions, a person's hearing may be protected for life.

In this regard, OSHA enforces a Hearing Conservation Program for employers to help control hearing loss injury in the workplace. In the Hearing Conservation Program, OSHA identifies five main requirements: noise exposure monitoring, audiogram testing, employee training, hearing protection devices, and recordkeeping. Audiogram testing, also commonly known as a hearing test, is typically required within the first six months of employment as a baseline test and then is typically required on an annual basis following the baseline test. Unfortunately, audiogram testing results often stay with the employer and do not get shared with future employers. This poses a gap in understanding the employee's true hearing health history as each employee often starts over with a new baseline audiogram test with their next employer. Additionally, some employers risk compliance and fail to perform the requisite audiogram testing for various reasons, such as the associated cost or inconvenience of scheduling testing for their employees.

While certain devices and methods for performing audiogram testing are known, it is believed that no one prior to the inventors has made or used the invention described in the appended claims.

### BRIEF DESCRIPTION OF THE DRAWINGS

The accompanying drawings, which are incorporated in and constitute a part of this specification, illustrate embodiments of the invention, and, together with the general description of the invention given above, and the detailed description of the embodiments given below, serve to explain the principles of the present invention.

FIG. 1 depicts a schematic view of an exemplary hearing health monitoring system;

FIG. 2 depicts an exemplary Digital Signal Processor (DSP) device and functionalities;

FIG. 3 depicts an exemplary infrastructure workflow of the hearing health monitoring system of FIG. 1;

FIG. 4 depicts an exemplary user interface of the hearing health monitoring system of FIG. 1;

FIG. 5 depicts an exemplary advanced hearing testing method relative to a standard hearing testing method;

FIG. 6 depicts exemplary protective eyewear with at least one DSP and microphone;

FIG. 7 depicts a schematic view of an exemplary noise mitigating system;

FIG. 8A depicts an exemplary results table that may be generated by the hearing health monitoring system of FIG. 1;

FIG. 8B depicts an exemplary results graph that may be generated by the hearing health monitoring system of FIG. 1;

FIG. 8C depicts an exemplary recorded ambient sound levels graph that may be generated by the hearing health monitoring system of FIG. 1; and

FIG. 8D depicts an exemplary event log that may be generated by the hearing health monitoring system of FIG. 1.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

In some instances, it may be desirable to provide a data capturing and mitigation system and method to prevent noise induced hearing loss through an audio digital signal processor and software. The present disclosure is directed generally to an instrument and software system that performs, processes, evaluates, retains, diagnoses, and predicts hearing acuity test results. Additional applications through the use of an Audio Digital Signal Processor and software infrastructure also apply. Such application can include the ability to proactively disrupt soundwaves reducing sound pressure intensity.

This instrument may be connected to cloud servers, application programming interface and web-based applications that evaluate, read, and retain current and historic audiometry results to learn, detect and predict future hearing acuity. Data such as cumulative noise and ototoxic particle exposure may be used to detect early signs of hearing loss. Additionally, data on exposure to sound frequency levels, pitch, impulse, impact or pressure levels can be used to determine early signs. This may provide the end-user with the ability to diagnose current hearing threshold levels and to uncover early signs of hearing loss before it happens.

The instruments, systems, and methods disclosed herein also have applications for mitigating sound sources. Such applications may include evaluating, retaining, learning, detecting, and predicting sound patterns to proactively disburse inverse soundwaves that may ultimately reduce ambient noise and pressure levels.

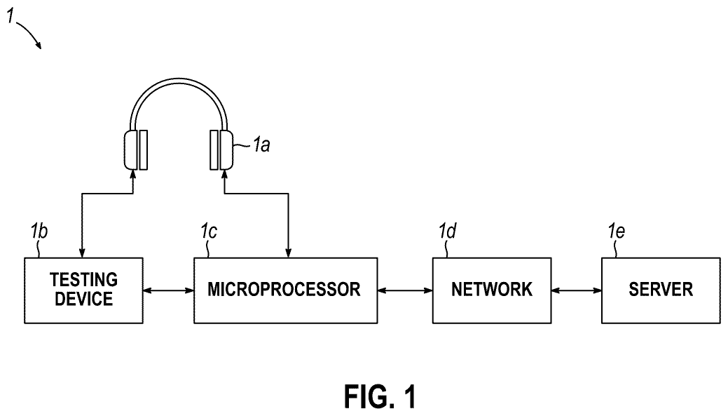

In some instances, it may be desirable to connect an acoustic Digital Signal Processor (DSP) or similar microprocessor to an instrument and control input and output data. FIG. 1 depicts a system (1) including a sound emitter in the form of headphones (1a), a testing device (1b), a DSP or microprocessor (1c), a network (1d), and a server (1e). The arrows shown in FIG. 1 represent bi-directional communication between various components of the illustrated system (1). In the example shown, DSP (1c) is integrated with testing device (1b) for an audiometric test. Audiometric tests may detect sensorineural hearing loss, which may include damage to the nerve or cochlea, and/or conductive hearing loss, which may include damage to the eardrum or the auditory ossicle bones. During an audiometry evaluation, a variety of tests may be performed. These may include a pure tone audiometry test, which measures the softest (e.g., least audible) sound that a person can hear. During such a test, headphones, such as headphones (1a), may be worn by the person receiving the test over the person's ears.

In this regard, headphones (1a) may be used to play sounds to test a person's hearing level. Such testing can include a pure tone audiometry test to measure the softest, or lowest audio sound that the person can hear, or any other suitable testing for determining the person's hearing level.

Testing device (1b) includes the audiometry controlling equipment, which may be provided in the form of any one or more of an audiometer, microprocessor audiometer, computer, laptop, tablet, phone or other instruments used to perform audiometric testing. Testing device (1b) may be configured to transmit recorded sounds such as pure tones, speech, or other sounds to headphones (1a). For example, testing device (1b) may be configured to transmit sounds at fluctuating frequencies and/or intensities to headphones (1a) while headphones (1a) are being worn by the person receiving the test. Testing device (1b) may also be configured to record the person's responses to produce an audiogram, which may include a graph showing the results of the tested person's hearing threshold sensitivity. These results may be displayed (e.g., via a graphical user interface of testing device (1b)) in measurements of decibels (dB) for loudness and/or Hertz (Hz) for frequencies. It will be appreciated that established normal hearing range may be between about 250 Hz and about 8,000 Hz at about 25 dB or lower.

As shown, DSP or microprocessor (1c) may be in operative communication with testing device (1b) and/or headphones (1a). For example, DSP or microprocessor (1c) may be integrated with testing device (b) and/or headphones (1a) through any one or more of the internet, USB, HDMI, Bluetooth, or any other suitable connectivity protocols. In some versions, DSP or microprocessor (1c) may be directly incorporated into testing device (1b). In addition, or alternatively, DSP or microprocessor (1c) may be directly incorporated into headphones (1a), such as for facilitating direct and/or remote audiometric testing. Connecting DSP (1c) to testing device (1b) and/or headphones (1a) transforms traditional testing instruments into “smart” or internet connected instruments which allows the instrument to push and receive information over a network (1d). Such information may include remote calibration, testing controls and data retained in server (1e). Furthermore, DSP (1c) may have the ability to convert analog data from traditional instruments into digital data.

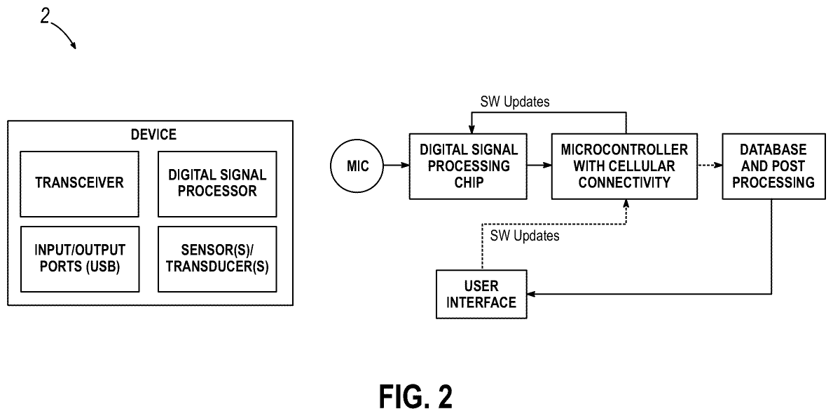

In some versions, DSP (1c) may control the input and output of ambient sound and pressure levels. It will be appreciated that DSP (1c) may replace traditional analog circuits to perform functions like A-weighting. In addition, or alternatively, DSP (1c) may be capable of communicating back and forth over a data bus with other components, thereby enabling multiple audio channels to be read without using additional general-purpose input/output (GPIO) resources. In some versions, DSP (1c) may be configured to perform real time frequency analysis that may be used to determine whether there has been a change to a machine's noise signature. Such functionalities are described in greater detail below in connection with FIG. 2.

Network (1d) may include any suitable type of communication network for placing microprocessor (1c) in operative communication with the internet. For example, network (1d) may include any one or more of a cellular (e.g. LTE) network, Wi-Fi network, and/or an ethernet network. Microprocessor (1c) may thus be connected to the internet through network (1d).

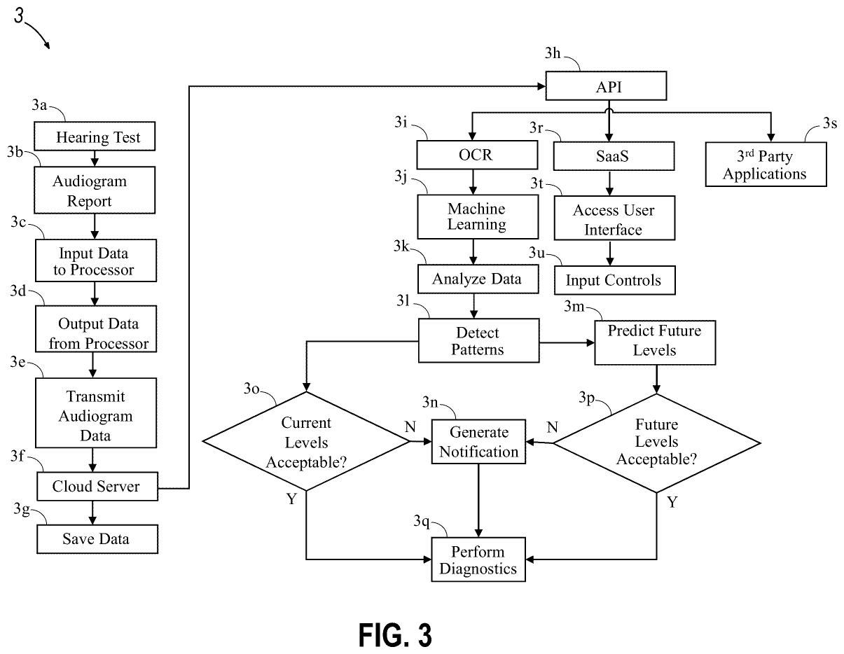

Server (1e) may include any suitable type of server, such as a cloud server. Network (1d) may be in operative communication with cloud server (1e), which may be configured to provide any one or more of data management, data storage, and/or recordkeeping of audiometry data (e.g., via cloud-based storage). In this regard, audiograms obtained via testing device (1b) and/or microprocessor (1c) may be sent through network (1d) to cloud server (1e). Cloud server (1e) may, in turn, be in operative communication with a computing interface such as that described below in connection with FIG. 3, which may include open application programming interface (API) such as that described below in connection with FIG. 3, and connectors, for example for providing a general layer on top of the cloud data. Any one or more applications and/or third party integrations may flow through the computing interface. In this regard, applications may include any one or more of user management applications, computer, tablet, mobile device, artificial intelligence applications, robotic programming automation, remote calibration applications, data visibility applications, optical character recognition, analytics applications, date and time applications, personal identification applications, and/or reporting applications.

As shown in FIG. 2, an exemplary testing device (2) may include a transceiver, a processor(s), microprocessor(s), DSP, input/output ports (e.g., USB ports), and/or one or more sensors/transducers, cellular or other internet connecting boards. In addition, or alternatively, testing device (2) may include a microphone that measures ambient noise or sound levels (decibels) simultaneously during an audiometric test.

FIG. 2 also illustrates a testing device and software that can perform audiometric tests while simultaneously monitoring live ambient noise levels. The ambient noise is measured by a calibrated sound monitoring system and network. The sound is collected through a calibrated microphone with class one or class two sound level meter standards. The entire audiometric and ambient sound recording device is connected to the internet and has the ability to receive remote updates through the internet such as firmware and device calibrations.

Referring now to FIG. 3, an exemplary method (3) for monitoring the hearing health of a person (also referred to as a test subject) that may be performed by the system (1) shown in FIG. 1 begins at step (3a), whereat an audiometric hearing test is performed on the person, such as via headphones (1a). Method (3) proceeds from step (3a) to step (3b), at which an audiogram results are completed or simultaneously while the test is being administered for the person based on the performed audiometric hearing test. Method (3) proceeds from step (3b) to step (3c), whereat the audiogram report image or digital report is inputted into a processor, such as processor (1c). This may be performed either automatically or manually through scanning an image of the report. Method (3) proceeds from step (3c) to step (3d), at which the audiogram is outputted from the processor. Method (3) proceeds from step (3d) to step (3e), at which the audiogram data is transmitted by a transceiver, such as through a network, to a server, such as a cloud server. Method (3) proceeds from step (3e) to step (3f) at which the cloud server manages various input and output data, including the audiogram data received from the transceiver. Method (3) proceeds from step (3f) to step (3g) at which the audiogram report data (e.g., both current/new audiogram report data and historical audiogram report data) is saved on a secure server location, such as cloud-based storage.

In the example shown, method (3) also proceeds from step (3f) to step (3h) at which the cloud server accesses an application programming interface (API) for interacting with other software and/or applications. In this regard, method (3) of the present example proceeds from step (3h) to step (3i) at which the audiogram data is inputted in real-time into an image/data reading application, such as an Optical Character Recognition (OCR) application, which may visually read the audiogram image results. As shown, method (3) proceeds from step (3i) to step (3j), at which the audiogram data is inputted into a machine learning algorithm (e.g., connected to the cloud server of FIG. 1), via the API for learning audiogram patterns and predicting future audiogram patterns. In some versions, method (3) may directly proceed from step (3h) to step (3j), bypassing step (3i), such as in cases where the audiogram data is processed digitally with or without the use of DSP (1c) such that visual reading of the audiogram image results may not be needed.

As shown, method (3) proceeds from step (3j) to step (3k), at which the machine learning algorithm analyzes the new audiogram data. Method (3) proceeds from step (3k) to step (3l) at which the machine learning algorithm detects patterns by comparing the new audiogram data against historical audiogram data (e.g., retrieved from the data saved at step (3g)). Method (3) proceeds from step (3l) to step (3m), at which a future hearing acuity/audiogram prediction is performed. This prediction may be comprised from the audiogram results of step (3b) compared to the historical audiogram results retrieved from the data saved at step (3g). Additionally, step (3m) may incorporate additional data that may also be retained in the same cloud-based storage as that in which the data is saved in step (3g). For example, such additional data may include personal information such as medical history, gender, age, ethnicity, geography, job description, and other factors that may be considered as affecting hearing acuity. In addition, or alternatively, step (3m) may compare audiogram data and unique personal data to multiple processed audiograms (e.g., via prior performances of step (3c)) stored historically (e.g., from prior performances of step (3g)) for prior test subjects having similar personal data (e.g., medical history, gender, age, ethnicity, geography, job description, etc.). Information from step (3r), described below, such as noise exposure information, or sound intensity scores may also be used. In addition, or alternatively, third party applications from step (3s), also described below, such as additional data and analytics applications may also be included in the predicted calculation of step (3m). In some versions, step (3m) may also use information from the end user obtained via input controls at step (3u), also described below.

Method (3) also proceeds from step (3l) to step (3o) at which the machine learning algorithm determines whether the current audiogram readings are acceptable. For example, step (3o) may include determining whether the audiogram results are within a predetermined range, such as a Standard Threshold Shift (STS). In this regard, an STS is currently defined in the occupational noise exposure standard 29 CFR 1910.95(g)(10)(i) as a change in hearing threshold, relative to the baseline to the audiogram for that employee, of an average of 10 dB or more at 2000 Hz, 3000 Hz, and 4000 Hz in one or both ears. The current STS calculation and requirements may be determined through calculating the difference between the annual audiogram and the baseline audiogram at 2,000 Hz, 3,000 Hz, and 4,000 Hz to determine a decibel shift value for each frequency; summing the decibel shift values for each frequency; and dividing the sum by 3. A first example of how to perform this calculation using a first exemplary set of data is provided in the table below.

| | Annual | Baseline | |
|---|---|---|---|
| Frequency | Audiogram | Audiogram | Annual − Baseline |
| 2,000 Hz | 15 dB | 10 dB | 15 dB − 10 dB = 5 dB |
| 3,000 Hz | 20 dB | 15 dB | 20 dB − 15 dB = 5 dB |
| 4,000 Hz | 30 dB | 15 dB | 30 dB − 15 dB = 15 dB |

The average change for the above example is equal to (5 dB+5 dB+15 dB)/3=(25 dB)/3=8.33 dB. Since 8.33 dB is less than 10 dB, STS has not occurred. Thus, the current audiogram readings may be considered acceptable for this example.

A second example of how to perform this calculation using a second exemplary set of data is provided in the table below.

| | Annual | Baseline | |
|---|---|---|---|
| Frequency | Audiogram | Audiogram | Annual − Baseline |
| 2,000 Hz | 15 dB | 5 dB | 15 dB − 5 dB = 10 dB |
| 3,000 Hz | 20 dB | 10 dB | 20 dB − 10 dB = 10 dB |
| 4,000 Hz | 30 dB | 10 dB | 30 dB − 10 dB = 20 dB |

The average change for this example is equal to (10 dB+10 dB+20 dB)/3=(40 dB)/3=13.33 dB. Since 13.33 dB is greater than 10 dB, STS has occurred. Thus, the current audiogram readings may be considered unacceptable for this example.

If the machine learning algorithm determines that the current audiogram readings are unacceptable (e.g., by determining that the hearing results are above the standard threshold shift) then method (3) proceeds from step (30) to step (3n), at which an automated detection warning is generated and communicated to the user. Method (3) proceeds from step (3n) to step (3q) at which various diagnostics are performed as described below. If the machine learning algorithm determines that the current audiogram readings are acceptable, then method (3) proceeds directly from step (3o) to step (3q) for such diagnostics.

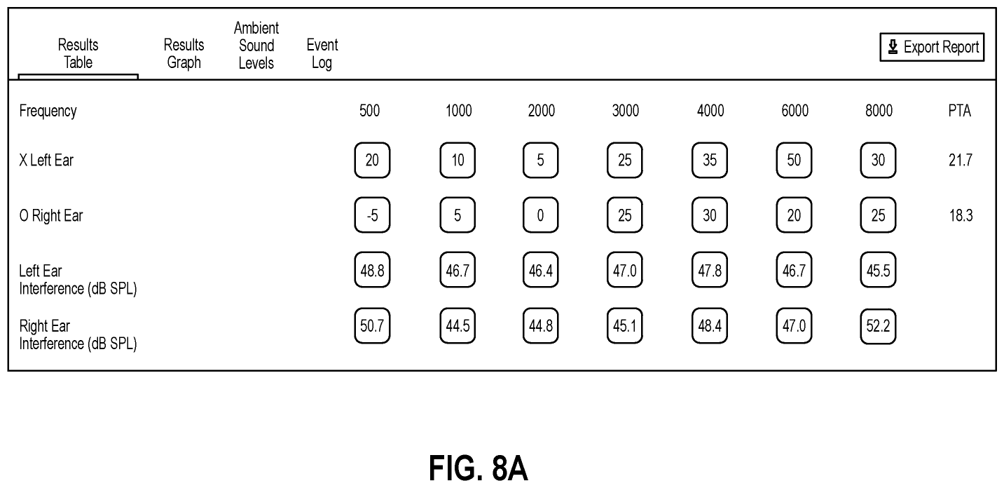

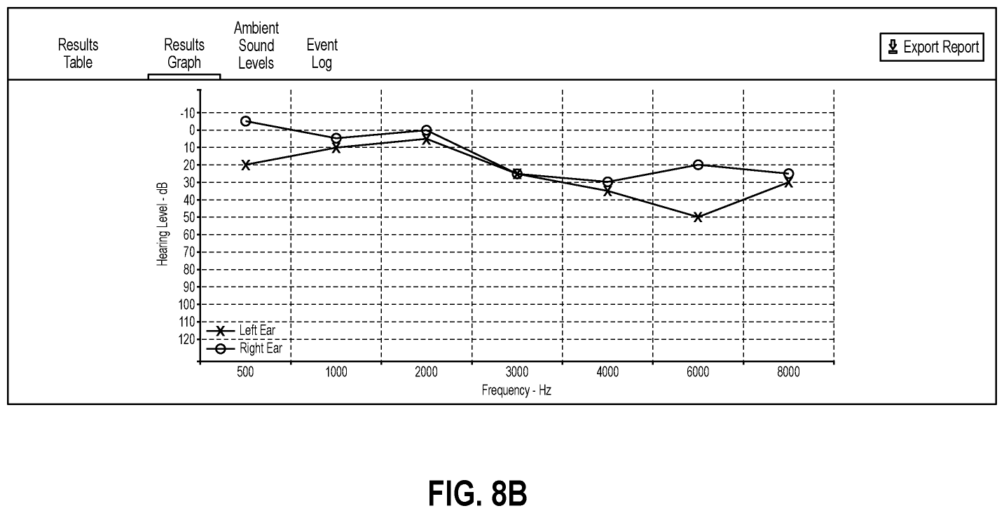

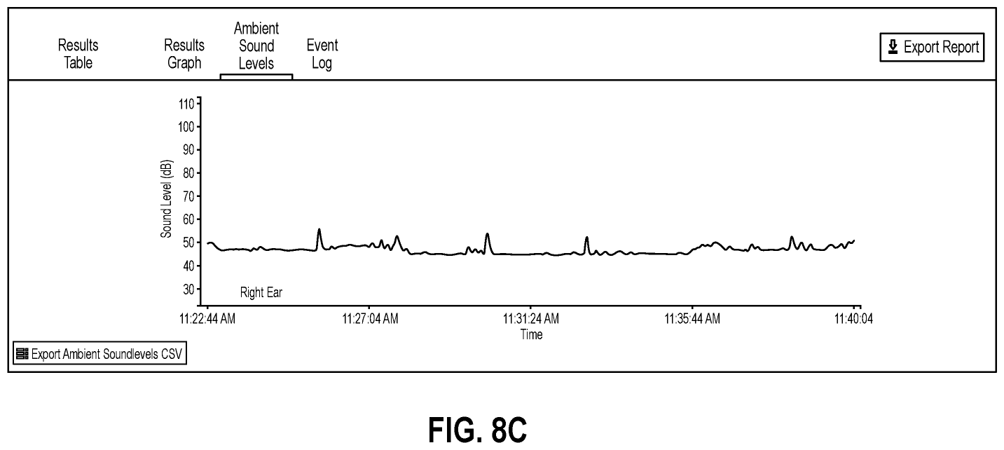

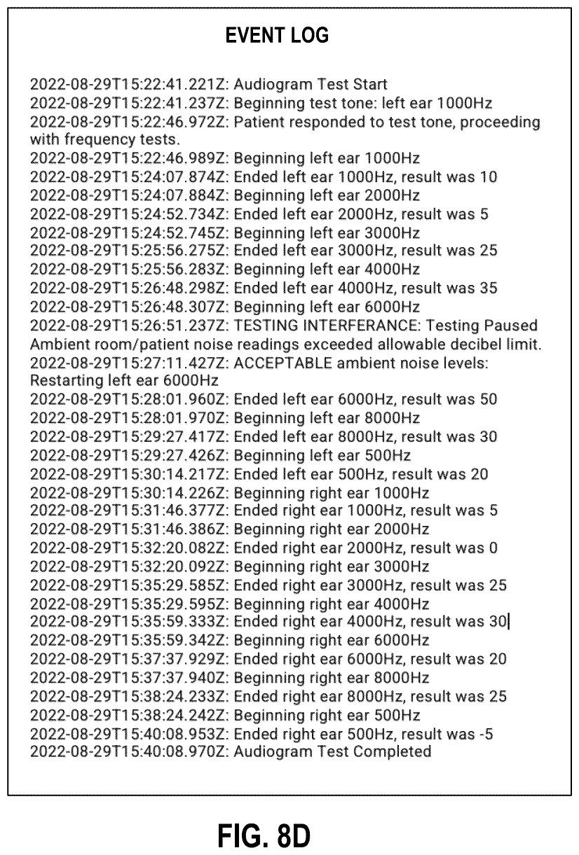

Further regarding step (3o), active environmental factors may also contribute to an acceptable test result or not. Such factors may include active or real-time ambient noise level measurements recorded during an audiometric test. FIGS. 8A-8D reflect an example of a completed audiometric test with integrated noise levels (dB or SPL) monitored, measured, and recorded simultaneously throughout the entire audiometric test. The external or ambient sound is measured and/or recorded by a calibrated (class 1 or class II) microphone or octave band analyzer. FIG. 8D shows an event log in which the left ear 6000 Hz tone was interrupted. The microphone detected ambient noise levels above the allowable threshold such as 60 decibels at the time the 6000 Hz tones were being administered. The test paused and restarted after ambient noise levels reached an acceptable range again. Interference such as high sound levels during an audiometric test can cause inaccurate patient responses. Integrating active noise monitoring levels throughout a patient audiometric test provides critical data for more accurate and consistent results.

As noted above, method (3) also proceeds from step (3l) to step (3m), at which the machine learning algorithm may predict future STS's based on the detected audiogram patterns. Method (3) proceeds from step (3m) to step (3p) at which the machine learning algorithm determines whether predicted future STS levels are acceptable, such as whether the predicted future STS levels are within a predetermined range. For example, the predicted STS/hearing acuity levels may be considered “normal” if they are less than 25 db HL; “mild” if they are between 25 dB HL and 40 dB HL; “moderate” if they are between 41 dB HL and 65 dB HL; “severe” if they are between 66 dB HL and 90 dB HL; and “profound” if they are more than 90 dB HL. If the machine learning algorithm determines that the predicted STS/hearing acuity levels are unacceptable, such as any of “mild,” “moderate,” “severe,” or “profound,” then method (3) proceeds to step (3n), at which the automated notification warning is generated and communicated to the user. As noted above, method (3) proceeds from step (3n) to step (3q) for diagnostics. If the machine learning algorithm determines that the predicted future hearing acuity levels are acceptable, such as “normal,” then method (3) proceeds directly from step (3p) to step (3q) for such diagnostics.

At step (3q), current and predicted Standard Threshold Shift and hearing acuity level data evaluated through the machine learning algorithm are reported for a full diagnosis and analysis, and the data is inputted back into the machine learning algorithm for continued learning of rules, patterns, and behaviors associated with the STS/audiogram levels, and is so transmitted to the cloud server of step (3f) via the computing interface of step (3r) for data record keeping in the cloud-based storage of step (3g) and/or for other purposed described below.

Method (3) also proceeds from step (3h) to step (3r), at which the cloud server of step (3f) interacts, via the application computing interface of step (3h), with software-as-a-service (SaaS), such as a web-based application, which may include any one or more of displaying current and/or historic data (e.g., noise exposure measurements provided via the system of U.S. Pub. No. 2022/0286797, audiometry testing controls, audiogram results, standard threshold shifts, predicted hearing threshold shift, warning notifications, user controls, diagnostic and reporting capabilities), enabling the management of current, historic and predictive hearing acuity level recordings and data analytics, and/or allowing a user to view and/or control certain operating controls or other parameters of audiometric testing, reading, managing, etc.

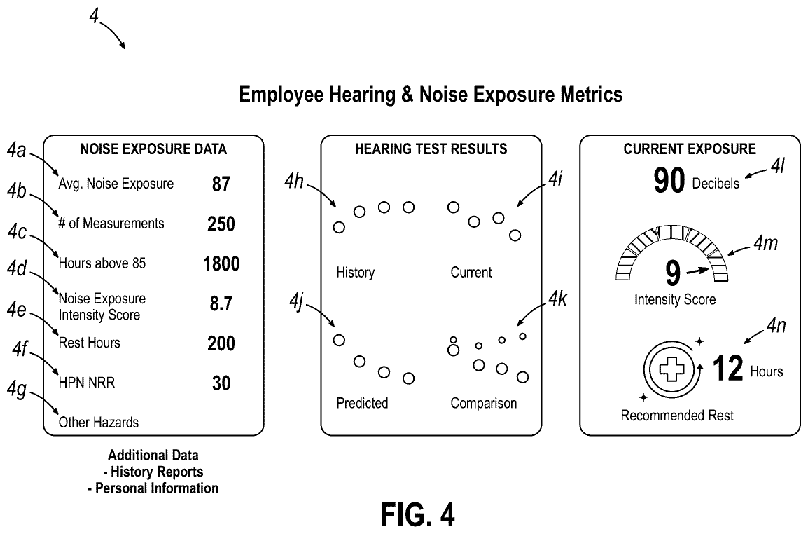

The current STS diagnosis method as explained above are restricted in data. Incorporating data such as cumulative noise and ototoxic exposure, data from previous audiograms and other metrics such as any one or more of those identified in FIG. 4 may provide a more accurate depiction of one's hearing health.

| | Annual | Baseline | |
|---|---|---|---|
| Frequency | Audiogram | Audiogram | Annual − Baseline |
| 2,000 Hz | 15 dB | 10 dB | 15 dB − 10 dB = 5 dB |
| 3,000 Hz | 20 dB | 15 dB | 20 dB − 15 dB = 5 dB |
| 4,000 Hz | 30 dB | 15 dB | 30 dB − 15 dB = 15 dB |

The average change for the above example is equal to (5 dB+5 dB+15 dB)/3=(25 dB)/3=8.33 dB. Since 8.33 dB is less than 10 dB, STS has not occurred. Thus, the current audiogram readings may be considered acceptable for this example.

Using the same readings listed above but this time incorporating additional data retained in server (3f) reflected in FIG. 3.

For example purposes only: (5 dB+5 dB+15 dB)/3=(25 dB)/3=8.33+Hearing Loss Decline Rate Algorithm=11.33.

Since 11.33 dB is greater than 10 dB, STS has occurred. Thus, the current audiogram readings may be considered unacceptable for this example.

Incorporating the Hearing Loss Decline Rate may also be used for intervention purposes. (5 dB+5 dB+15 dB)/3=(25 dB)/3=8.33+Hearing Loss Decline Rate Algorithm=Estimated shift to 11.33 in 6 months. Example intervention methods: Prevent or delay decline through limiting exposure to hazardous noise, wearing hearing aids, wearing hearing protection and other mitigation methods.

Method (3) proceeds from step (3r) to step (3t), at which the user accesses a user interface (e.g., via the SaaS of step (3r)), such as remotely, to conduct, operate, diagnose, view, monitor and manage audiometric testing and/or equipment, which may include testing device (1b) and/or DSP (1c). For example, the user may access the user interface to send decibel and frequency tones to testing device (1b). In this regard, method (3) proceeds from step (3t) to step (3u), at which various controls are inputted to a processor, such as processor (1c), such as via cloud server (1e). This may include conducting pre-set, artificial intelligent or live audiometric testing, in-person or from a remote location. In addition, or alternatively, such controls may include any one or more of software updates, remote calibration, on/off commands, decibel/frequency intensity signals and tones, and other operating and reporting commands (e.g., inputting date/time, personal data information, etc.). When the audiometric test is conducted in this manner via step (3t) and step (3u), the testing results may be processed as described herein (e.g., beginning at step (3a)).

Method (3) also proceeds from step (3h) to step (3s), at which cloud server (1e) interacts, via the computing interface of step (3h), with additional applications and integrations, which may include any associated third party applications.

While method (3) has been described as being performed in a particular order, it will be appreciated that various portions of method (3) may be performed in orders different from that described, and that certain portions may be omitted from method (3) in some versions.

Referring now to FIG. 4, an exemplary user interface (4) of system (1) includes a plurality of indicia (4a, 4b, 4c, 4d, 4e, 4f, 4g, 4h, 4i, 4j, 4k, 4l, 4m, 4n) for visually communicating various types of data or other information to provide an in-depth view of a person's noise exposure and/or hearing health. In this regard, research studies show that over exposure to noise will lead to hearing loss. However, such research studies generally do not quantify the amount of noise exposure that leads to hearing loss. There is also a lack of mass and granular noise exposure data that can be used to determine a person's true amount of noise exposure. Therefore, there is a wide range of permissible noise exposure limits from reputable organizations and government bodies. For example, based on the decibel level of 85 dB the World Health Organization recommends no more than 1 hour of noise exposure, while the National Institute of Safety & Health recommends no more than 8 hours, and the Occupational Safety and Health Organization recommends no more than 16 hours at 85 dB. In addition to these guidelines having a wide range of varying permissible noise exposure limits, these are also blanket guidelines that are not tailored to particular individuals. Thus, they do not take into account the fact that every person has a different sensitivity to noise such that every person may be susceptible to different types or degrees of ear anatomy damage caused by noise. System (1) may be configured to provide individualized data regarding a person's noise exposure and/or hearing health, and recommendations tailored to suit that particular person.

In the example shown, first through seventh indicia (4a, 4b, 4c, 4d, 4e, 4f, 4g) visually communicate the person's noise exposure data and metrics. More particularly, first indicia (4a) visually communicates the person's average noise exposure in a numerical form. In this regard, the person's average noise exposure may include the person's average noise time-weighted exposure level, and may be calculated with known equations based on time and decibel levels. For example, first indicia (4a) in FIG. 4 shows the average noise exposure as 87 dB.

Second indicia (4b) visually communicates the person's amount of measurements in a numerical form, which may include the number of days or recordings that the person monitored the person's noise exposure. For example, second indicia (4b) in FIG. 4 shows the number of measurements as 250.

Third indicia (4c) visually communicates the person's cumulative amount of time spent being exposed to noise above a predetermined threshold in a numerical form. For example, third indicia (4c) in FIG. 4 shows the cumulative amount of time that the person has spent being exposed to noise above a threshold of 85 dB as 1800 hours. It will be appreciated that a threshold other than 85 dB may be used, and that a unit of time other than hours may be used, such as minutes.

Fourth indicia (4d) visually communicates the person's noise exposure intensity/sensitivity grade/score in a numerical form. In this regard, the occupational health and safety administration has a blanket policy for allowable noise exposure limits. Medical experts acknowledge that each individual has a unique sensitivity to noise. A number of different factors can determine sensitivity, such as genetics, previous hearing damage, age, ototoxic chemicals, and other factors. This is a recently-developed category that gives an accurate depiction of the particular person's noise exposure. Calculated into this on-going algorithm is unique personal information such as cumulative noise exposure, age, gender, previous hearing acuity metrics, and other uniquely identifying information. Furthermore, additional data from other individuals may be factored into the equation for comparison and accuracy purposes. For example, fourth indicia (4d) in FIG. 4 shows the noise exposure intensity/sensitivity grade/score as 8.7. This exemplary score may be assigned to a 45-year-old male who is exposed to a cumulative average of 83 decibels daily. Factoring his gender, age, noise exposure data, hearing acuity results along with (or without) comparison to known data of other individuals, this person's noise exposure intensity grade may be increased by 4, thereby giving him a total score of 8.7. This grade is uniquely calculated based on each individual or subject. As noted above, genetics, previous hearing damage, and/or other factors may contribute to the person's sensitivity to noise.

Fifth indicia (4e) visually communicates a preventative health metric including an amount of rest time recommended for the person to avoid noise in a numerical form. The amount of rest time recommended may be based on the noise exposure intensity grade. For example, fifth indicia (4e) in FIG. 4 shows the amount of rest time as 200 hours. As another example, if the person reaches the allowable noise exposure limit after 4 hours of a work shift, then the amount of rest time recommended for the person to avoid noise may include the remainder of the person's work shift. As another example, if the person is within or exceeds the noise exposure limit, then the amount of rest time recommended for the person to avoid noise may be a predetermined amount of hours before the person may be exposed to hazardous noise levels again.

Sixth indicia (4f) visually communicates the person's hearing protection devices noise reduction rating (HDP NRR) in a numerical form, which indicates the person's hearing protection and noise attenuation. For example, sixth indicia (4f) in FIG. 4 shows the person's HDP NRR as 30.

Seventh indicia (4g) visually communicates other potential hazards to the person. In this regard, the software interface may not be limited to the data and metrics described above. In some versions, any one or more additional metrics such as air quality, ototoxic chemicals, anti-noise metrics, noise attenuation data, and other contributing factors may be displayed.

In the example shown, eighth through eleventh indicia (4h, 4i, 4j, 4k) visually communicate the person's hearing test results. More particularly, eighth indicia (4h) visually communicates the person's hearing test history in a graphical form, which represents the person's historic hearing acuity. This may include one historic audiogram or a cumulative report of multiple historic audiograms.

Ninth indicia (4i) visually communicates the person's current or most recent audiogram results in a graphical form. These results may be obtained in the manner described above via system (1) and/or method (3), for example.

Tenth indicia (4j) visually communicates the person's predicted future audiogram results in a graphical form. These results may be obtained in the manner described above via system (1) and/or method (3), for example. In addition, or alternatively, these results may include the person's noise exposure data and noise intensity grades to estimate future hearing loss or hearing acuity.

Eleventh indicia (4k) visually communicates the person's predicted comparison in a graphical form, which represents the person's predicted hearing acuity without any changes to the person's lifestyle versus the person's predicted hearing acuity with intervention. Such intervention may include any one or more of wearing hearing protection devices, wearing hearing aids, limiting noise exposure, increasing rest between noise exposure, etc.

In the example shown, twelfth through fourteenth indicia (4l, 4m, 4n) visually communicate the person's current noise exposure. Information regarding the person's current noise exposure may be provided via another system (not shown), that is configured to monitor real-time and predicted sound level tracing. Such a system may be configured and operable in accordance with at least some of the teachings of U.S. Pub. No. 2022/0286797, entitled “Smart Sound Level Meter for Providing Real-Time Sound Level Tracing,” published on Sep. 8, 2022, the disclosure of which is incorporated by reference herein in its entirety. More particularly, twelfth indicia (4l) visually communicates the person's latest noise exposure reading in a numerical form, which represents the person's current or most recent noise time weighted average reading. For example, twelfth indicia (4l) in FIG. 4 shows the person's latest noise exposure reading as 90 dB.

Thirteenth indicia (4m) visually communicates the person's intensity/hearing loss score in an animated gauge and/or numerical form, which represents the person's current or most recent noise intensity grade. For example, thirteenth indicia (4m) in FIG. 4 shows the person's intensity/hearing loss score as 9.

Fourteenth indicia (4n) visually communicates a recommended amount of rest in numerical form and/or other recommended intervention to prevent further damage to the person's hearing based on the current noise exposure data and intensity grade. For example, fourteenth indicia (4n) in FIG. 4 shows the recommended amount of rest as 12 hours.

Any one or more of the metrics identified in FIG. 4 can be used to compute a hearing loss decline rate algorithm. For example, patterns detected from the person's hearing test results as identified by eighth through eleventh indicia (4h, 4i, 4j, 4k), the person's noise exposure data and metrics as identified by first through seventh indicia (4a, 4b, 4c, 4d, 4e, 4f, 4g), and/or the person's current noise exposure as identified by twelfth through fourteenth indicia (4l, 4m, 4n) can determine the pace and timeline one may lose their hearing. As noted above, 30%-50% of hair cells are damaged or destroyed before hearing loss is detected. This algorithm can provide an estimation of the remaining healthy hair cells or rate at which one is damaging their hair cells based on personal and exposure data.

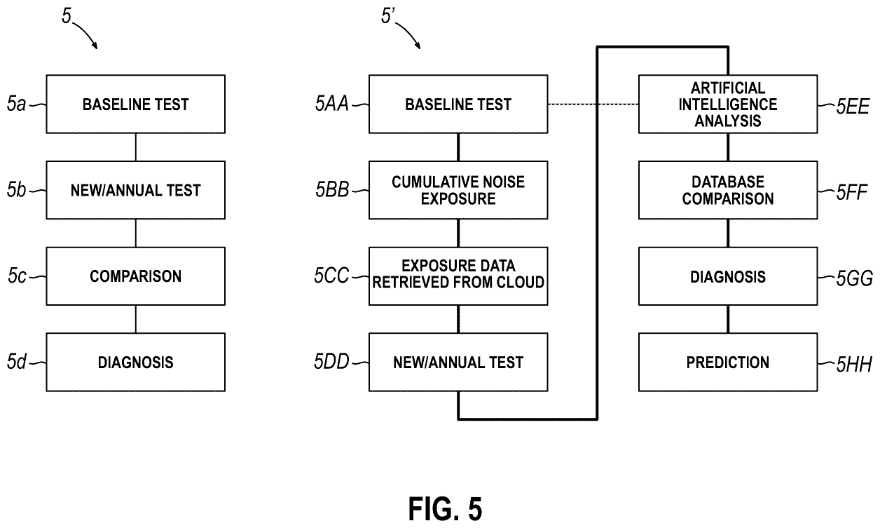

Referring now to FIG. 5, an advanced testing method (5′) is depicted relative to a standard testing method (5). In the occupational space, method (5) includes step (5a), at which a baseline test is performed within the first 6 months of employment. Hearing Standard Threshold Shifts will be based on this baseline line test. As noted above, audiogram records often do not get transferred from one employer to the next which leaves a major gap in one's hearing health history. Indeed, the National Academy of Sciences identified the lack of hearing loss surveillance data as a major shortcoming of the NIOSH Hearing Loss Research Program. At step (5b), a new/annual test is performed. For example, employers may be required to have their employees perform a new/annual audiogram test. At step (5c), a comparison is performed. As noted above, the baseline test is compared to the new test to calculate the Standard Threshold Shift. At step (5d), a diagnosis is provided.

Method (5′) includes step (5AA), at which the baseline test data is digitally recorded or converted to digital data. At step (5BB), noise and hazardous exposure such as ototoxic hazards are monitored throughout the year. At step (5CC), exposure data is provided from server (1e). At step (5DD), the new/annual test includes noise and hazardous exposure data as an additional factor in calculating hearing acuity. At step (SEE), an artificial intelligence review is performed, wherein a machine learning algorithm identifies changes and learns decline rate. At step (5FF), a data comparison is performed, wherein artificial intelligence compares testing results to mass hearing loss surveillance data. At step (5GG), a diagnosis is provided, wherein traditional hearing shift results are identified with the addition of step (5HH), at which prediction of loss of hair cells, hearing loss decline rate and estimated hearing loss timeline are also provided.

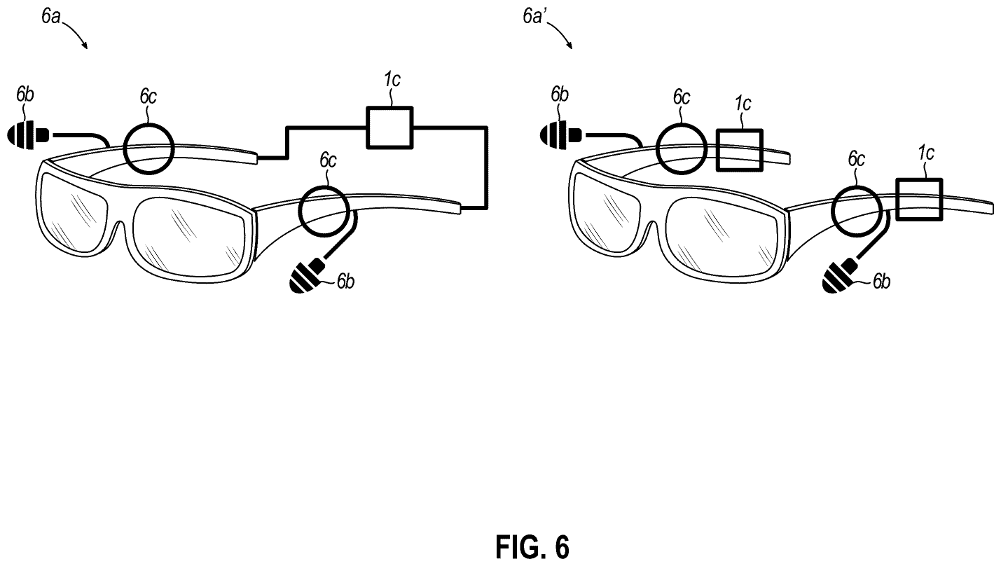

Referring now to FIG. 6, two examples of protective eyewear (6a, 6a′) are shown as being equipped with one or more DSPs (1c). Protective eyewear is commonly worn in the industrial space and is often required to be worn. Statistics show that protective eyewear has higher user adoption than hearing protective devices. In some cases, hearing protection such as protective earmuffs or earplugs (6b) may be incorporated into protective eyewear (6a, 6a′). This allows eye and ear protection along with noise exposure data through one protective piece of equipment. It will be appreciated that eyewear (6a, 6a′) are configured and operable to perform the same functions described above for instrument (1a) in connection with FIG. 1. Additional testing such as remote, virtual or digital vision tests may be performed using eyewear (6a, 6a′). Vision tests infrastructure may follow similar cloud server and methods as explained in prior figures for hearing tests. In the examples shown, eyewear (6a, 6a′) are also equipped with one or more microphones (6c), which may be configured and operable in accordance with at least some of the teachings of U.S. Pub. No. 2022/0286797, entitled “Smart Sound Level Meter for Providing Real-Time Sound Level Tracing,” published on Sep. 8, 2022, the disclosure of which is incorporated by reference herein in its entirety.

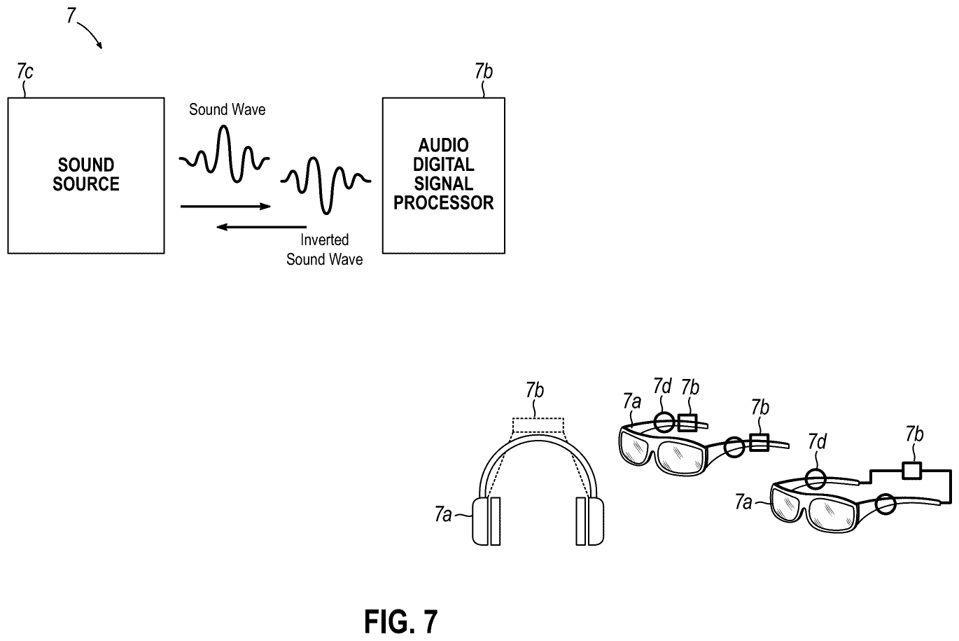

In some instances, it may be desirable to proactively disrupt soundwaves with inverted soundwaves to reduce decibel or sound pressure levels. FIG. 7 depicts a system (7) including at least one form of personal protective equipment (PPE) such as earmuffs and/or glasses (7a), an audio digital signal processor (7b) affixed to PPE (7a), and a sound source (7c). DSP (7b) may the same as DSP (1c) described above. DSP (7b) may be configured to transmit a soundwave inversion to counteract one or more soundwaves generated by sound source (7c). To effectively transmit the correct soundwave inversion the transmitting device must determine the sound source or sound wave pattern generated by sound source (7c) prior to the soundwaves reaching the person wearing PPE (7a). This determination may be performed by DSP (7b). Furthermore, DSP (7b) may be in operative communication with another system (not shown), that is configured to monitor real-time and predicted sound level tracing, to thereby provide DSP (7b) with historic decibel and sound pressure level data. Such a system may be configured and operable in accordance with at least some of the teachings of U.S. Pub. No. 2022/0286797, entitled “Smart Sound Level Meter for Providing Real-Time Sound Level Tracing,” published on Sep. 8, 2022, the disclosure of which is incorporated by reference herein in its entirety. This data can be used by DSP (7b) to predict sound wave patterns that allow DSP (7b) to proactively transmit the correct inverted wave to reduce sound intensity and pressure levels. In the examples shown, glasses (7a) are also equipped with one or more microphones (7d), which may be configured and operable in accordance with at least some of the teachings of U.S. Pub. No. 2022/0286797, entitled “Smart Sound Level Meter for Providing Real-Time Sound Level Tracing,” published on Sep. 8, 2022, the disclosure of which is incorporated by reference herein in its entirety.

Referring now to FIGS. 8A-8D, an example of a completed audiometric test and the recorded patient responses aligned with active noise monitoring metrics are shown. FIG. 8A shows a results table that includes the patient's left and right ear hearing acuity results from 500 to 8000 hz (hertz). While not shown, additional hertz such as 5000, 7000, 10,000 and more may be included in audiometric tests. The results table of FIG. 8A also reflects the recorded ambient or room decibel level recorded at the time of each respected ear and frequency. FIGS. 8B and 8C show the metrics from FIG. 8A in a graph illustration. More particularly, FIG. 8B shows a results graph including the patient's hearing acuity results and FIG. 8C shows the recorded ambient noise levels recorded during the test. FIG. 8D depicts a comprehensive event log that details live data recorded during an audiometric test. Shown in the description and in the event log of FIG. 8D is an example of live “testing interference.” The testing device and software detected noise levels loud enough that it could affect the patient's response for the left ear at 6000 Hz. The device and software automatically paused and restarted playing tones when the ambient noise levels reached an acceptable testing level:
2022-08-29T15:26:51.237Z: TESTING INTERFERANCE: Testing Paused

Ambient room/patient noise readings exceeded allowable decibel limit.

2022-08-29T15:27:11.427Z: ACCEPTABLE ambient noise levels:
Restarting left ear 6000 Hz

Combination of patient response, comparison to historic audiograms, real-time noise levels and other contributing factors are used to determine an accuracy or confidence score of audiometric testing results. This will help prevent inaccurate tests from being accepted that have unusual output or odd trends compared to historical records. An audiometer with real-time noise monitoring that can adjust the frequency threshold levels for ambient noise levels in the room. For explanatory purposes, an ambient noise level of 30 decibels is recorded during 2000 hz tone, the patient's response is 5 but when the patient takes a second audiometric test, the noise level increases to 43 decibels during 2000 hz tones and the patient's recorded response is 25. The confidence score would be low because the ambient noise levels increased by 13 decibels from test 1 to test 2. If the test 2 had consistent ambient noise levels with test 1 then the confidence score would be high.

### EXAMPLES

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A hearing health monitoring system comprising: (a) a sound emitter configured to play sounds to test a person's hearing level; (b) a testing device configured to transmit the sounds to the sound emitter; and (c) a processor in operative communication with at least one of the testing device or the sound emitter, wherein the processor is configured to send and receive data associated with testing the person's hearing level to and from a cloud server over a network.

### Example 2

The hearing health monitoring system of Example 1, wherein the sound emitter includes headphones.

### Example 3

The hearing health monitoring system of any of Examples 1 through 2, wherein the testing device includes an audiometer.

### Example 4

The hearing health monitoring system of any of Examples 1 through 3, wherein the processor includes a Digital Signal Processor (DSP).

### Example 5

The hearing health monitoring system of any of Examples 1 through 4, wherein the processor is integrated with the sound emitter.

### Example 6

The hearing health monitoring system of any of Examples 1 through 5, wherein the processor is integrated with the testing device.

### Example 7

The hearing health monitoring system of any of Examples 1 through 6, wherein the processor is integrated with protective eyewear.

### Example 8

The hearing health monitoring system of any of Examples 1 through 7, wherein the data includes an audiogram report.

### Example 9

The hearing health monitoring system of any of Examples 1 through 8, wherein the processor is configured to provide a notification in response to a determination that the person's current hearing level is outside of a predetermined range.

### Example 10

The hearing health monitoring system of any of Examples 1 through 9, wherein the processor is configured to provide a notification in response to a determination that the person's estimated future hearing level is outside of a predetermined range.

### Example 11

A method for monitoring hearing health comprising: (a) performing a hearing test on a human subject; (b) generating audiogram data for the human subject based on the hearing test; (c) transmitting the audiogram data for the human subject to a cloud server over a network; and (d) analyzing the audiogram data via a machine learning algorithm.

### Example 12

The method of Example 11, further comprising detecting patterns by comparing the audiogram data for the human subject against historical data via the machine learning algorithm.

### Example 13

The method of Example 12, wherein the historical data includes data associated with the human subject.

### Example 14

The method of any of Examples 12 through 13, wherein the historical data includes data associated with other human subjects.

### Example 15

The method of any of Examples 11 through 14, further comprising determining whether the audiogram data for the human subject is acceptable via the machine learning algorithm.

### Example 16

The method of Example 15, further comprising generating a notification in response to a determination that the audiogram data for the human subject is not acceptable.

### Example 17

The method of any of Examples 11 through 16, further comprising estimating future audiogram data for the human subject via the machine learning algorithm.

### Example 18

The method of Example 17, further comprising generating a notification in response to a determination that the estimated future audiogram data for the human subject is not acceptable.

### Example 19

The method of any of Examples 11 through 18, further comprising performing diagnostics with the audiogram data for the human subject via the machine learning algorithm.

### Example 20

The method of Example 19, wherein performing diagnostics includes inputting processed data back into the machine learning algorithm for continued learning of patterns associated with the audiogram data.

### Example 21

The method of any of Examples 11 through 20, further comprising monitoring real-time ambient noise while performing the hearing test, and automatically pausing and restarting the hearing test in response to the monitored real-time ambient noise.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A hearing health monitoring system comprising:
(a) a sound emitter configured to play sounds to test a person's hearing level;
(b) a testing device configured to transmit the sounds to the sound emitter; and
(c) a processor in operative communication with at least one of the testing device or the sound emitter, wherein the processor is configured to send and receive data associated with testing the person's hearing level to and from a cloud server over a network.

2. The hearing health monitoring system of claim 1, wherein the sound emitter includes headphones.

3. The hearing health monitoring system of claim 1, wherein the testing device includes an audiometer.

4. The hearing health monitoring system of claim 1, wherein the processor includes a Digital Signal Processor (DSP).

5. The hearing health monitoring system of claim 1, wherein the processor is integrated with the sound emitter.

6. The hearing health monitoring system of claim 1, wherein the processor is integrated with the testing device.

7. The hearing health monitoring system of claim 1, wherein the processor is integrated with protective eyewear.

8. The hearing health monitoring system of claim 1, wherein the data includes an audiogram report.

9. The hearing health monitoring system of claim 1, wherein the processor is configured to provide a notification in response to a determination that the person's current hearing level is outside of a predetermined range.

10. The hearing health monitoring system of claim 1, wherein the processor is configured to provide a notification in response to a determination that the person's estimated future hearing level is outside of a predetermined range.

11. A method for monitoring hearing health comprising:
(a) performing a hearing test on a human subject;
(b) generating audiogram data for the human subject based on the hearing test;
(c) transmitting the audiogram data for the human subject to a cloud server over a network; and
(d) analyzing the audiogram data via a machine learning algorithm.

12. The method of claim 11, further comprising detecting patterns by comparing the audiogram data for the human subject against historical data via the machine learning algorithm.

13. The method of claim 12, wherein the historical data includes data associated with the human subject.

14. The method of claim 12, wherein the historical data includes data associated with other human subjects.

15. The method of claim 11, further comprising determining whether the audiogram data for the human subject is acceptable via the machine learning algorithm.

16. The method of claim 15, further comprising generating a notification in response to a determination that the audiogram data for the human subject is not acceptable.

17. The method of claim 11, further comprising estimating future audiogram data for the human subject via the machine learning algorithm.

18. The method of claim 17, further comprising generating a notification in response to a determination that the estimated future audiogram data for the human subject is not acceptable.

19. The method of claim 11, further comprising performing diagnostics with the audiogram data for the human subject via the machine learning algorithm.

20. The method of claim 19, wherein performing diagnostics includes inputting processed data back into the machine learning algorithm for continued learning of patterns associated with the audiogram data.

21. The method of claim 11, further comprising monitoring real-time ambient noise while performing the hearing test, and automatically pausing and restarting the hearing test in response to the monitored real-time ambient noise.
